(19) United States
(12) Patent Application Publication
(10) Pub. No.: US 2010/0184872 A1
(43) Pub. Date: Jul. 22, 2010

(54) PREPARATION OF FISCHER-TROPSCH CATALYSTS

(75) Inventors: Sigrid Eri, Ranheim (NO); Erling Rytter, Trondheim (NO); Oyvind Borg, Trondheim (NO); Alejandro Antonini, Stockton on Tees (GB); Kate Elizabeth McCulloch, Cleveland (GB)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(73) Assignees: GTL.F1 AG, Zurich (CH); JOHNSON MATTHEW PLC, London (GB)

(21) Appl. No.: 12/525,070

(22) PCT Filed: Jan. 29, 2008
(86) PCT No.: PCT/GB08/00300

§ 371 Date: Mar. 26, 2010

(30) Foreign Application Priority Data
Jan. 30, 2007 (GB) 0701740.3

(51) Int. Cl. B01J 23/889; B01J 19/00; B01J 8/00; C07C 27/00

(52) U.S. Cl. 518/700; 502/332; 422/198; 422/187; 518/715

(57) ABSTRACT

A method of producing a catalyst for use in a Fischer-Tropsch synthesis reaction. The method comprises the steps of: impregnating a catalyst support material with an active cobalt catalyst component to form a catalyst precursor; and calcining the catalyst precursor in an atmosphere of a dry calcining gas.

### PRIORITY CLAIM

The present application is a National Phase entry of PCT Application No. PCT/GB2008/000300, filed Jan. 29, 2008, which claims priority from Great Britain Application Number 0701740.3, filed Jan. 30, 2007, the disclosures of which are hereby incorporated by reference herein in their entirety.

### TECHNICAL FIELD

The present invention is concerned with the preparation of Fischer-Tropsch catalysts, and the catalysts so produced.

### BACKGROUND

The Fischer-Tropsch (FT) reaction for conversion of synthesis gas, a mixture of CO and hydrogen, possibly also containing essentially inert components like CO₂, nitrogen and methane, is commercially operated over catalysts containing the active metals iron (Fe) or cobalt (Co). However, the iron catalysts exhibit a significant shift reaction, producing more hydrogen in addition to CO₂ from CO and steam. Therefore the iron catalyst will be most suited for synthesis gas with low H₂/CO ratios (<1.2), e.g. from coal or other heavy hydrocarbon feedstock, where the ratio is considerably lower than the consumption ratio of the FT reaction (2.0-2.1).

In systems where the H₂/CO ratio in the synthesis gas is higher, e.g. where the hydrocarbon feedstock is methane, cobalt is the preferred catalyst. The present invention is particularly concerned with Co-based catalysts in various embodiments.

Normally, the active FT metal is dispersed on a solid, porous support. In this way, a large portion of the Co is exposed as surface atoms where the reaction can take place. The support can be alumina, titania or silica, but in fact, other oxides such as zirconia, magnesia, zeolites have been used, as has carbon.

To enhance the catalyst performance, e.g. by facilitating the reduction of cobalt oxide to cobalt metal prior to the FT synthesis, it is common to add different promoters. In this regard, rhenium, ruthenium, platinum, iridium and other transition metals can all be beneficial.

A number of different impregnation procedures are known in the art which use various solvents and chemicals, and which are suitable. However, in the present specification, the examples involve melt impregnation or incipient wetness impregnation of cobalt nitrate hexahydrate (Co(NO₃)₂.6H₂O) onto the support. It is well known that the impregnation method may influence the dispersion of the active metal (cobalt) and hence the catalytic activity. Generally, after impregnation, the impregnated catalyst is dried, typically at 80-120° C., to remove water from the catalyst pores, and then calcined typically at 200-450° C., e.g. at 300° C. for 2-16 h.

The conditions during calcination may also have a significant effect on the final catalyst properties. According to EP-A-0 421 502, the activity may be improved, if during calcination, the catalyst is exposed to an atmosphere containing large amounts of nitrogen oxides. In contrast, however, van de Loosdrecht et al. (Topics in Catalysis, Vol. 26, 2003, pp. 121-127) recorded high cobalt metal surface areas and high catalytic activities when the concentration of nitrogen oxides and water was kept low during calcination.

### SUMMARY

According to embodiments of the invention, there is provided a method of producing a catalyst for use in a Fischer-Tropsch synthesis reaction, comprising: impregnating a catalyst support material with an active metal-based catalyst component to form a catalyst precursor; and calcining the catalyst precursor in an atmosphere of a dry calcining gas.

### BRIEF DESCRIPTION OF THE DRAWING

FIG. 1 is a graph of cobalt surface areas obtained for catalysts in example embodiments.

### DETAILED DESCRIPTION

It is an object of embodiments of the invention to provide an improved F-T catalyst.

Embodiments of a method of producing a catalyst for use in a Fischer-Tropsch synthesis reaction are provided, comprising impregnating a catalyst support material with an active metal-based catalyst component to form a catalyst precursor; and calcining the catalyst precursor in an atmosphere of a dry calcining gas.

In an embodiment, the water content of the calcining gas is reduced to produce a vapor pressure<14.5 mm Hg. In an embodiment, the water content of the gas used in the calcination has been reduced by one or more steps of condensing, absorption, membrane separation, dilution and pressure reduction. In an embodiment, the calcining is carried out by passing the dry calcining gas through or over a bed of catalyst precursor particles and, in an embodiment, the active metal-based catalyst component is cobalt. The dry calcining gas may be an inert gas, such as nitrogen, or an oxygen containing gas such as air. In an embodiment, the impregnation includes impregnation with a catalyst promoter component, such as a rhenium component and the catalyst, after calcination and reduction, contains an amount of rhenium in the range 0.1 to 2 wt % as a promoter, assuming complete reduction of rhenium.

In an embodiment, the precursor is subjected to a drying step, prior to the calcining. In an embodiment, the drying step is carried out at a temperature in the range 80 to 160° C., such as 110 to 150° C., for a period of 0.2 to 10 hours, such as 0.5 to 4 hours. In an embodiment, the catalyst is subjected to a reduction step after the calcining step. In an embodiment, the catalyst has a metal surface area that is increased by at least 10% as compared to a catalyst prepared by calcinations in air without water vapor pressure reduction.

In an embodiment, the impregnating is achieved by means melt impregnation or incipient wetness impregnation techniques. Other suitable impregnation techniques include: precipitation from solution; precipitation/deposition; chemical vapor deposition; and vacuum impregnation. In an embodiment, the support material is impregnated with cobalt nitrate hexahydrate (Co(NO₃)₂.6H₂O). In an embodiment, the support material is impregnated with an aqueous solution of cobalt nitrate hexahydrate, containing an amount of cobalt to give a catalyst with a cobalt loading in the range 10 to 50 wt %, preferably 10 to 25 wt %, assuming complete reduction of cobalt. In an embodiment, the aqueous solution also contains an amount of rhenium to give a catalyst with a rhenium loading in the range 0.1 to 2 wt %, such as 0.2 to 1 wt %, as a promoter, assuming complete reduction of rhenium.

In an embodiment, the support material is alumina, titania, silica, zirconia, magnesia or zeolite. In one embodiment, the material is γ-alumina, though any other form of alumina alone or in combination with a spinel-type aluminate, e.g. α-alumina together with nickel aluminate, may be used in other embodiments.

In an embodiment, the support material contains at least 10% by weight of a spinel compound composed of a divalent metal and aluminium. In an embodiment, the divalent metal is nickel or zinc. In an embodiment, the support material, prior to impregnation, has a specific surface area in the range 20 to 500 m²/g, preferably 30 to 200 m²/g. In an embodiment, the support material, prior to impregnation, has a pore volume greater than 0.1 cm³/g, such as greater than 0.2 cm³/g.

We have found, surprisingly, that the humidity of the gas to which the material is exposed during calcination can have an effect of the F-T activity of the resulting catalyst. Hence, we have found that exposure of the catalyst precursor to dry gases, i.e. gases in which the water content has been reduced, during the calcination step, offers improved Fisher-Tropsch catalysts. The gas used during calcination may be an oxygen-containing gas, e.g. air, or an inert gas such as nitrogen, or mixtures of these. Air is preferred in one embodiment.

In an embodiment, the calcining is conducted at a temperature in the range 150 to 450° C., such as 200 to 350° C., for a period of 1 to 16 hours, such as 1 to 10 hours. In the present invention the water content of the gas used in the calcination step may be reduced by one or more steps of condensing, absorption, membrane separation, dilution or pressure reduction, to have a water vapor pressure of, for example, <14.5 mm Hg. In one embodiment, the gas passed over the catalyst precursor during the calcining step has a humidity defined by a water vapor pressure of <5 mm Hg, such as <1 mm Hg, providing a dew point of <1.3° C., such as <−17.3° C. respectively.

The term “passed over the catalyst precursor” means that the gas is passed over the individual particles of the precursor. Conveniently, this is achieved by passing the gas through a bed of the precursor particles. The gas may be preheated prior to being passed through the precursor particles. In one embodiment, the calcination is performed on a fluidised bed of catalyst precursor in fluidised bed calciner.

In an embodiment, the dry calcining gas contains less than 500 vppm of water. Thus, the calcining gas may contain, in the range of 20 to 1000 vppm of water, such as between 50 and 500 vppm of water. The dry calcining gas may be obtained by cooling the gas, thereby condensing water or by using a sorption material to remove water. The reduced water vapor pressure in the calcining gas may be obtained by mixing an un-dried gas with a stream of dried gas, by compressing the gas to condense water and separating the condensed water, by passing the gas through a membrane that physically separates the water from the gas, or by performing the calcination under vacuum, such as at a pressure of less than 0.2 atm.

In an embodiment, the gas is passed through a bed of catalyst precursor particles. In an embodiment, the gas a flow rate giving a gas hourly space velocity (GHSV)>400 hr⁻¹, such as >1000 hr⁻¹.

In an embodiment, the surface area of the cobalt in the catalyst, after calcination and reduction is in the range 5 to 30% m²/g catalyst, such as 10 to 25 m²/g.

The invention also extends to a catalyst produced by the above method. In an embodiment, the cobalt content of the catalyst is from 10 to 40% by weight, such as from 10 to 25% by weight.

The invention also extends to the use of such a catalyst in an F-T synthesis reaction. In an embodiment, the reaction is carried out in a slurry bubble column reactor. In an embodiment, H₂ and CO are supplied to a slurry in the reactor, the slurry comprising the catalyst in suspension in a liquid including the reaction products of the H₂ and CO, the catalyst being maintained in suspension in the slurry at least partly by the motion of the gas supplied to the slurry.

The invention also extends to embodiments of a process for the production of hydrocarbons which comprise subjecting H₂ and CO gases to a Fischer-Tropsch synthesis reaction in a reactor in the presence of the catalyst.

In an embodiment, the reaction is a three-phase reaction in which the reactants are gaseous, the product is at least partially liquid and the catalyst is solid. In an embodiment, the reaction is carried out in a slurry bubble column reactor. In an embodiment, the H₂ and CO are supplied to a slurry in the reactor, the slurry comprising the catalyst in suspension in a liquid including the reaction products of the H₂ and CO, the catalyst being maintained in suspension in the slurry at least partly by the motion of the gas supplied to the slurry.

In an embodiment the reaction temperature is in the range 190 to 260° C., such as 210 to 240° C., and the pressure is in the range 10 to 60 bar, such as 15 to 35 bar. In an embodiment, the ratio H₂/CO of the gases supplied to the Fischer-Tropsch synthesis reactor is in the range 1.1 to 2.2, such as 1.5 to 1.95, and the superficial gas velocity in the reactor is in the range 5 to 60 cm/s, such as 20 to 40 cm/s.

In an embodiment, the product of the Fischer-Tropsch synthesis reaction is subsequently subjected to post-processing. The post-processing may include de-waxing, hydro-isomerisation, hydro-cracking and combinations of these.

The invention also extends to an apparatus for carrying out embodiments of the method of the invention, comprising a calcination vessel through which the catalyst precursor and calciner gas may pass having a catalyst precursor inlet, a calcined catalyst precursor outlet, a calciner gas inlet and a calciner gas outlet, and in which the calciner gas inlet is operatively connected to gas drying apparatus. In an embodiment, the gas drying means comprises condensing means that cool the gas to below the dew point of the water contained therein to condense and optionally to freeze the water and thereby separate the condensed or frozen water from the air fed to the calcination. Alternatively, the gas drying apparatus comprises a vessel having a wet gas inlet and a dry gas outlet and an absorption material, such as a particulate zeolite, disposed between the wet gas inlet and the dry gas outlet.

Porous catalyst support materials typically have specific surface areas between 30 and 500 m²/g. These supports can be prepared by spray drying techniques of an appropriate solution in order to obtain essentially spherical particles of appropriate size, e.g. 80% in the range between 30-150 μm. After spray-drying, the material is calcined at a high temperature to give the appropriate support crystal size and pore structure.

It is important that the total pore volume of the support is sufficiently high, above 0.1 cm³/g or better, above 0.2 cm³/g, or even better above 0.6 cm³/g in various embodiments. The pore volume is often measured by nitrogen adsorption/desorption. This method does not take into account large pores where a mercury porosimeter is more relevant. A less accurate, but more practical parameter is the measured water absorptivity, which can be directly correlated with the amount of cobalt that can be impregnated on the catalyst by the incipient wetness procedure. A high pore volume will give a light material suitable for operation in a slurry environment and ease the impregnation by minimising the number of impregnation steps required. At the same time the support, and the final catalyst, should have sufficient strength for extended operation of months and years with minimal attrition of the materials. This can be tested in a slurry environment or by the ASTM method applicable for testing FCC (fluid catalytic cracking) catalysts.

The invention may be carried into practice in various ways and some embodiments will now be specifically described in the following non-limiting examples.

The starting alumina material used for all catalysts in the present invention was Puralox SCCa γ-Al₂O₃ from Sasol.

### Example 1

### Effect of Air Circulation

A catalyst precursor was prepared by one-step incipient wetness impregnation of γ-Al₂O₃ (BET surface area=187 m²/g, Pore volume=0.73 cm³/g) with an aqueous solution of cobalt nitrate hexahydrate. The sample contained a nominal amount of 20 wt % cobalt and 1 wt % rhenium, as calculated assuming reduced catalysts with complete reduction of cobalt. The actual metal loading as determined by XRF (x-ray fluorescent spectroscopy) or ICP (inductively coupled plasm spectrometry) may vary up 10%, e.g. for a catalyst with nominal loading of 20 wt %, the actual amount cobalt can vary between 18 and 22 wt % of the total reduced catalyst weight. The effect of air humidity for the catalytic properties was tested by varying the calcination arrangement. One (sample 1) was calcined in a crucible located inside an oven. The air flow was not passed directly through the sample. The other sample (sample 2) was calcined as a bed of catalyst particles inside a glass reactor located inside the same oven and subjected to a continuous flow of air. In this case, the air flowed directly through the sample. The calcination temperature and time were 300° C. and 10 h, respectively. For Sample 1, ambient air surrounded the crucible before heating, whereas dry instrument air at room temperature was passed via a tube through the wall of the oven for Sample 2. In the latter case, the water content of the air was 500 vppm.

The catalytic properties of sample 1 and 2 are given in Table 1. The high activity of sample 2 compared to sample 1 was related to the lower amounts of water and NO in the calcination atmosphere for the latter sample. By passing dry air directly through the sample, we believe that the calcination decomposition products were removed very efficiently. The calcination effect is probably related to different cobalt surface areas after calcination since it is generally believed that the FT-reaction is non-structure sensitive.

TABLE 1
Catalytic properties of catalysts

| Catalyst | Relative activity | Relative selectivity |
|---|---|---|
| 1 | 1.53 | 0.908 |
| 2 | 1.91 | 0.909 |

### Example 2

### Effect of Air Humidity

The effect of air humidity for the cobalt surface area was tested for another series of catalysts. A catalyst precursor was prepared by one-step incipient wetness impregnation of a γ-Al₂O₃ support (of the same kind as used in Example 1) with an aqueous solution of cobalt nitrate hexahydrate. The sample contained a nominal amount of 20 wt % cobalt. After impregnation, the catalyst precursor was dried at 110° C. for 3 h and subsequently split into samples of 1.2 g. These samples were calcined for 10 h under different conditions (air at 30 ml/min; air at 50 ml/min; 50% air/50% steam at 30 ml (min) and at different temperatures. The water content in the air was <40 vppm.

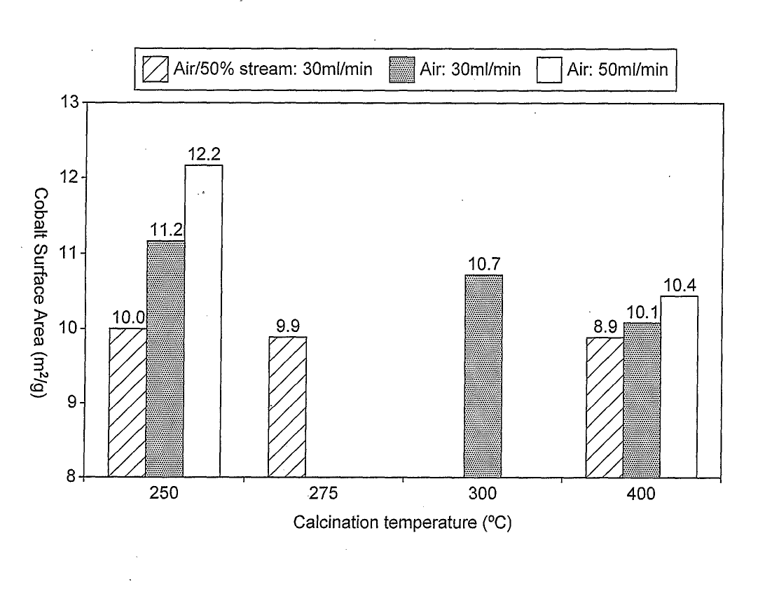

The cobalt surface areas obtained for the catalysts of this series are given in FIG. 1. Calcination in humid air clearly resulted in lower cobalt surface area than calcination in dry air. Increasing the air flow rate and therefore decreasing the water content during calcinations is clearly beneficial. The difference was largest for the lowest calcination temperatures. The reason for the big difference was probably different Co₃O₄ crystallite sizes prior to in situ reduction. X-ray diffraction data gave a crystallite size of 13.2 and 6 nm, respectively.

### Example 3

### Effect of Air Humidity

The catalysts of this series contain a nominal amount of 20wt % Co and 0.5 wt % Re. Cobalt nitrate hexahydrate was heated above the melting point and mixed with ammonium perrhenate. The mixture was then added to the support. Before impregnation, the catalyst support (BET surface area=175 m2/g, Pore volume=0.71 cm3/g) was pre-calcined at 500° C.

The freshly prepared catalysts were dried at a temperature of 110° C. After impregnation and drying, calcination was carried out in a fluidised bed reactor. In all cases, the calcination temperature and time were 250° C. and 2 hours, respectively. The effect of humidity during calcination was studied by feeding air flows of different humidity to the calcination reactor. Both dry instrument air (dew point<−30° C.) and air with higher levels of humidity were used as calcination agents. The water content (and air dew point) was controlled by saturating air with water in a heated bubbler and mixing with dry air. In comparison to the present invention illustrated by experiments A, B, C & D, experiments E, F & G were run with humidity's above 14.5 mmHg. The prepared catalysts are summarised in Table 2, which clearly shows that the air humidity level is important for the catalytic activity. Fisher-Tropsch activities and selectivity's were measured relative to experiment G. Calcination in air with a dew point of 0° C. or below resulted in relative activities in the range 1.10 to 1.14 in the Fisher-Tropsch synthesis. The activities after calcination in humid air were lower when the inlet air dew point was 17° C. or higher.

TABLE 2
Experimental conditions and catalytic data.

| | | | Calcination | | Catalytic | |
|---|---|---|---|---|---|---|
| | Catalyst | | conditions | | performance | |
| Cata- | composition | | | Space | | Relative |
| lyst | Co | Re | Dew point | velocity | Relative | C5+ |
| name | (wt %) | (wt %) | air (° C.) | Litre/g Co/hr | activity | selectivity |
| A | 20 | 0.5 | −30 | 22.4 | 1.14 | 1.01 |
| B | 20 | 0.5 | −30 | 22.5 | 1.10 | 1.02 |
| C | 20 | 0.5 | 0 | 20.3 | 1.12 | 1.01 |
| D | 20 | 0.5 | 5 | 20.3 | 1.04 | 1.01 |
| E | 20 | 0.5 | 11 | 22.5 | 0.99 | 1.01 |
| F | 20 | 0.5 | 12 | 22.5 | 0.98 | 1.00 |
| G | 20 | 0.5 | 30 | 23.9 | 1.00 | 1.00 |

Hydrogen Chemisorption

Hydrogen adsorption isotherms were recorded on a Micromeritics ASAP 2010 unit at 313 K. The samples were reduced in situ in flowing hydrogen at 623 K for 16 h. The temperature was increased by 1 K/min from ambient to 623 K. After reduction, the samples were evacuated at 623 K and cooled down under vacuum to 313 K. An adsorption isotherm was recorded in the pressure interval 20 to 510 mmHg. The amount of chemisorbed hydrogen was calculated by extrapolating the linear part of the isotherm to zero pressure. In order to calculate the cobalt surface area, it was assumed that two cobalt sites were covered by one hydrogen molecule and that the area of one cobalt atom is 6.62·10⁻²² m²/atom.

1-54. (canceled)

55. A method of producing a catalyst for use in a Fischer-Tropsch synthesis reaction, comprising:
impregnating a catalyst support material with an active metal-based catalyst component to form a catalyst precursor;
calcining the catalyst precursor in an atmosphere of a dry calcining gas; and
reducing a water content of the calcining gas to produce a vapor pressure of less than about 14.5 mm Hg.

56. The method of claim 55, wherein the water content of the calcining gas is reduced by a process selected from the group consisting of condensing, absorption, membrane separation, dilution and pressure reduction.

57. The method of claim 55, wherein the calcining is carried out by passing the dry calcining gas through or over a bed of catalyst precursor particles.

58. The method of claim 55, wherein the active metal-based catalyst component is cobalt.

59. The method of claim 55, wherein the dry calcining gas comprises one of an inert gas or air.

60. The method of claim 55, wherein the impregnating includes impregnation with a catalyst promoter.

61. The method of claim 60, wherein the promoter is rhenium and wherein the catalyst, after calcining and reducing, contains an amount of rhenium in a range of about 0.1 to 2 wt % as a promoter, assuming complete reduction of rhenium.

62. The method of claim 55, wherein the precursor is subjected to a drying step, prior to the calcining.

63. The method of claim 62, wherein the drying step is carried out at a temperature in the range of about 80 to 160° C. for a period of about 0.2 to 10 hours.

64. The method of claim 55, wherein the catalyst is subjected to a reduction step, after the calcining.

65. The method of claim 55, wherein a catalyst has a metal surface area that is increased by at least about 10% as compared to a catalyst prepared by calcination in air without water vapor pressure reduction.

66. The method of claim 55, wherein the impregnating is achieved by one of a melt impregnation technique or an incipient wetness impregnation technique.

67. The method of claim 66, wherein the support material is impregnated with an aqueous solution of cobalt nitrate hexahydrate (Co(NO₃)₂.6H₂O), whereby the catalyst contains an amount of cobalt in a range of about 10 to 50 wt % assuming complete reduction of cobalt.

68. The method of claim 67, wherein the aqueous solution also contains an amount of rhenium in order to produce in the catalyst a rhenium content in a range of about 0.1 to 2 wt % as a promoter, assuming complete reduction of rhenium.

69. The method of claim 55, wherein the support material is selected from the group consisting of alumina, titania, silica, zirconia, magnesia and zeolite.

70. The method of claim 69, wherein the support material is one of γ-alumina, α-alumina, or an alumina in combination with a spinel-type aluminate.

71. The method of claim 70, wherein the support material contains at least about 10% by weight of a spinel compound composed of a divalent metal and aluminium.

72. The method of claim 71, wherein the divalent metal is one of nickel or zinc.

73. The method of claim 55, wherein the support material, prior to impregnating, has a specific surface area in a range of about 20 to 500 m²/g.

74. The method of claim 55, wherein the support material, prior to impregnating, has a pore volume greater than about 0.1 cm³/g.

75. The method of claim 55, wherein the calcining is conducted at a temperature in a range of about 150 to 450° C.

76. The method of claim 55, wherein dry calcining gas passed over the catalyst precursor during the calcining has a humidity defined by a water vapor pressure of less than about 5 mm Hg.

77. The method of claim 55, wherein dry calcining gas passed over the catalyst precursor during the calcining has a humidity defined by a dew point of less than about 1.3° C.

78. The method of claim 55, wherein the dry calcining gas contains less than about 500 vppm of water.

79. The method of claim 55, wherein the dry calcining gas contains in a range of about 20 to 1000 vppm of water.

80. The method of claim 78, wherein a reduced water vapor pressure in the dry calcining gas is obtained by one of cooling the gas, thereby condensing water, or using a sorption material to remove water.

81. The method of claim 78, wherein a reduced water vapor pressure in the calcining gas is obtained by one of mixing an un-dried gas with a stream of dried gas or compressing the gas to condense water and separating the condensed water.

82. The method of claim 78, wherein a reduced water vapor pressure in the calcining gas is obtained by one of passing the gas through a membrane that physically separates the water from the gas or performing the calcination under vacuum at a pressure of less than about 0.2 atm.

83. The method of claim 76, wherein the dry calcining gas is passed through a bed of catalyst precursor particles.

84. The method of claim 55, wherein the dry calcining gas has a flow rate giving a gas hourly space velocity (GHSV) of greater than about 400 hr⁻¹.

85. The method of claim 64, wherein a specific surface area of cobalt in the catalyst, after calcining and reducing, is in a range of about 5 to 30 m²/g.

86. A catalyst for use in a Fischer-Tropsch synthesis reaction produced according to the method of claim 85.

87. The catalyst of claim 86, wherein a cobalt content of the catalyst is from about 10 to 50% by weight.

88. A method comprising using of a catalyst as claimed in claim 86 in a Fischer-Tropsch synthesis reaction.

89. The method of claim 88, wherein H₂ and CO are supplied to a slurry in a slurry bubble column reactor, the reactor containing a slurry comprising the catalyst in suspension in a liquid including reaction products of the H₂ and CO, the catalyst being maintained in suspension in the slurry at least partly by motion of gas supplied to the slurry.

90. A process for the production of hydrocarbons comprising subjecting H₂ and CO gases to a Fischer-Tropsch synthesis reaction in a reactor in the presence of a catalyst as claimed as in claim 86.

91. The process of claim 90, wherein the reaction is a three-phase reaction in which reactants are gaseous, a product is at least partially liquid and the catalyst is solid.

92. The process of claim 91, wherein the reaction is carried out in a slurry bubble column reactor.

93. The process of claim 92, wherein the H₂ and CO are supplied to a slurry in the reactor, the slurry comprising the catalyst in suspension in a liquid including reaction products of the H₂ and CO, the catalyst being maintained in suspension in the slurry at least partly by motion of gas supplied to the slurry.

94. The process of claim 93, wherein a reaction temperature is in a range of about 190 to 250° C.

95. The process of claim 93, wherein a reaction pressure is in a range of about 10 to 60 bar.

96. The process of claim 93, wherein a H₂/CO ratio of gases supplied to the reactor is in a range of about 1.1 to 2.2.

97. The process of claim 93, wherein a superficial gas velocity in the reactor is in a range of about 5 to 60 cm/s.

98. The process of claim 93, wherein the product of the Fischer-Tropsch synthesis reaction is subsequently subjected to post-processing.

99. The process of claim 98, wherein the post-processing is selected from the group consisting of de-waxing, hydro-isomerisation, hydro-cracking and combinations thereof.

100. An apparatus configured to carry out the method of claim 55 comprising:
a gas drying apparatus and a calcination vessel through which the catalyst precursor and calciner gas may pass the vessel and having a catalyst precursor inlet, a calcined catalyst precursor outlet, a calciner gas inlet and a calciner gas outlet, wherein the calciner gas inlet is operatively connected to the gas drying apparatus.

101. The apparatus of claim 100, wherein the gas drying apparatus comprises condensing means that cool the gas to below the dew point of the water contained therein to condense the water and thereby separate the condensed water from air fed to the calcination vessel.

102. The apparatus of claim 100, wherein the gas drying apparatus comprises an absorption vessel having a wet gas inlet and a dry gas outlet and an absorption material, disposed between the wet gas inlet and the dry gas outlet.

103. The apparatus of claim 102, wherein the absorption material is a particulate zeolite.
